(19) United States
(12) Patent Application Publication
(10) Pub. No.: US 2016/0206267 A1
(43) Pub. Date: Jul. 21, 2016

(54) IMAGE PROCESSING APPARATUS, IMAGE DISPLAY SYSTEM, IMAGING SYSTEM, IMAGE PROCESSING METHOD, AND PROGRAM

(71) Applicant: TERUMO KABUSHIKI KAISHA, Tokyo (JP)

(72) Inventors: Hijiri SHIMIZU, Ashigarakami-gun (JP); Junya FURUICHI, Ashigarakami-gun (JP); Koichi INOUE, Ashigarakami-gun (JP)

(73) Assignee: TERUMO KABUSHIKI KAISHA, Tokyo (JP)

(21) Appl. No.: 15/081,237

(22) Filed: Mar. 25, 2016

Related U.S. Application Data
(63) Continuation of application No. PCT/JP2014/004870, filed on Sep. 24, 2014.

(30) Foreign Application Priority Data
Sep. 26, 2013 (JP) 2013-200484

(51) Int. Cl. A61B 6/00; A61B 6/02

(52) U.S. Cl. CPC A61B 6/5217; A61B 6/463; A61B 6/54; A61B 6/487; A61B 6/504; A61B 6/466; A61B 6/02

(57) ABSTRACT

In an imaging system, a vascular image extending along a vascular length direction, which is obtained from multiple transverse cross-sectional images of a blood vessel imaged by inserting a probe into the blood vessel, and multiple fluoroscopic images of the blood vessel imaged while the probe is inserted into the blood vessel are collected. A first position and a second position along the vascular length direction, which are designated by a user on the displayed vascular image, are acquired. The vascular image and the fluoroscopic image indicating the first position and the second position are displayed.

### CROSS-REFERENCES TO RELATED APPLICATIONS

This application is a continuation of International Application No. PCT/JP2014/004870 filed on Sep. 24, 2014, and claims priority to Japanese Patent Application No. 2013-200484 filed on Sep. 26, 2013, the entire content of each of which is incorporated herein by reference.

### TECHNICAL FIELD

The present invention relates to an image processing apparatus, an image display system, an imaging system, an image processing method, and a program, and particularly relates to image display for medical diagnosis.

### BACKGROUND DISCUSSION

Known medical procedures involving, for example, intravascular delivery of a balloon or a stent using a catheter are usually carried out with reference to a diagnostic image. In one such surgical procedure using the catheter, a stenosis, an occlusion, or the like in the blood vessel is confirmed by observing fluoroscopic images successively captured by means of angiography (Angio), for example, an X-ray image. In recent years, manual skills have been widely used to confirm the stenosis or the occlusion by concurrently observing vascular cross-sectional images obtained from an intravascular ultrasound (IVUS) endoscope, an optical coherence tomography (OCT) apparatus, an optical frequency domain imaging (OFDI) apparatus which is an improved model of the OCT apparatus and which uses wavelength sweeping, or the like.

In such procedures, the fluoroscopic images and the cross-sectional images are used mainly to perform preoperative diagnosis or to confirm a postoperative treatment effect. For example, when spreading out a vascular stenosis area by inserting the stent into the blood vessel, a surgeon (operator) identifies the vascular stenosis area after confirming an overall shape of a targeted coronary artery from the X-ray image. Furthermore, the surgeon will recognize intravascular symptoms by using the cross-sectional image of the stenosis area, and will also thereby determine an indwelling position or a size of the stent.

As a display method of the obtained image, for example, JP-A-2013-116332 discloses a parallel display technique in which a radiographic image and an IVUS image are associated with each other.

### SUMMARY

In order to perform diagnosis with reference to a vascular diagnostic image, it is important to recognize which position in the entire blood vessel corresponds to the blood vessel displayed on a screen. While utilizing a structural feature of a bifurcated portion or the like of the blood vessel as a mark, a surgeon can estimate a vascular position displayed on the screen in his or her head. However, such estimation requires particular skill on the part of the surgeon. In addition, in a case where any such structural feature is absent, it can be quite difficult to estimate the position.

The present disclosure describes an interface which enables a user to easily recognize a positional relationship between a vascular position displayed on a vascular image and the entire blood vessel.

The present disclosure describes an image processing apparatus including image collection means for collecting a vascular image extending along a vascular length direction, which is obtained from multiple transverse cross-sectional images of a blood vessel imaged by inserting a probe into the blood vessel, and multiple fluoroscopic images of the blood vessel imaged while the probe is inserted into the blood vessel, designation acquisition means for acquiring a first position and a second position along the vascular length direction, which are designated by a user on the vascular image or the fluoroscopic image displayed on display means, anddisplay control means for causing the display means to display an image that is designated by the user and an image that is not designated by the user from the vascular image and the fluoroscopic image which show the first position and the second position. Such a configuration can help make it possible to easily recognize a positional relationship between a vascular position displayed on a vascular image and the entire blood vessel.

### BRIEF DESCRIPTION OF THE DRAWINGS

FIG. 1 is a schematic configuration diagram of an image processing apparatus according to an embodiment.

FIG. 2 is a schematic configuration diagram of an OFDI apparatus.

FIG. 3 is a flowchart of an image processing method according to the embodiment.

FIG. 4 illustrates a screen display example 1 according to the embodiment.

FIG. 5 illustrates a screen display example 2 according to the embodiment.

FIG. 6 illustrates a screen display example 3 according to the embodiment.

FIG. 7 illustrates a screen display example 4 according to the embodiment.

FIG. 8 illustrates another example of the screen display example 4 according to the embodiment.

FIG. 9 illustrates a screen display example 5 according to the embodiment.

FIG. 10 illustrates another example of the screen display example 5 according to the embodiment.

FIG. 11 illustrates a screen display example 6 according to the embodiment.

### DETAILED DESCRIPTION

Hereinafter, embodiments according to the present disclosure will be described with reference to the drawings. In the drawings, the same reference numerals and signs will be applied to the same or similar configurations.

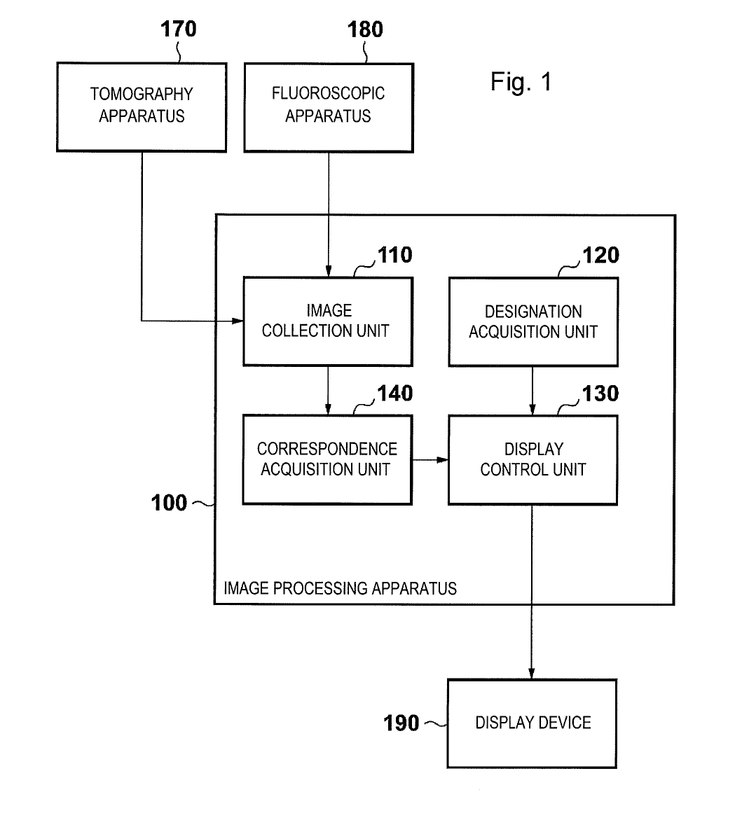

Hereinafter, an information processing apparatus according to Embodiment 1 will be described. FIG. 1 is a block diagram illustrating a configuration example of an image processing apparatus 100 according to the present embodiment embodied, for example, as a CPU. The image processing apparatus 100 according to the present embodiment includes an image collection unit 110, a designation acquisition unit 120, a display control unit 130, and a correspondence acquisition unit 140, embodied, for example, as software modules executed by the CPU corresponding to the image processing apparatus 100. In addition, the image processing apparatus 100 is connected to a tomography apparatus 170, a fluoroscopic apparatus 180, and a display device 190. The image processing apparatus 100 and the display device 190 configure an image display system. In addition, the image processing apparatus 100, the tomography apparatus 170, and the fluoroscopic apparatus 180 configure an imaging system.

The image collection unit 110 collects a vascular image extending along a vascular length direction (vascular major axis direction) and multiple vascular fluoroscopic images. The vascular image is obtained from multiple tomographic images, specifically transverse cross-sectional images of the blood vessel imaged by inserting a probe into the blood vessel, and includes a vascular axial cross-sectional image and a vascular 3D image. The vascular axial cross-sectional image represents a cross section along the vascular length direction, and the vascular 3D image represents a vascular shape at each position along the vascular length direction. The axial cross-sectional image or the 3D image can be reconstructed to include vascular transverse cross-sectional images. For example, the vascular 3D image can be reconstructed by extracting intravascular wall portions from the vascular transverse cross-sectional images and stacking the extracted inner wall portions along positions in the vascular length direction. In addition, if the vascular 3D image is cut out in the vascular length direction, the vascular axial cross-sectional image can be generated.

According to the present embodiment, the image collection unit 110 collects multiple vascular transverse cross-sectional images, and reconstructs a vascular image extending along the vascular length direction. However, the image collection unit 110 may collect the vascular image extending along the vascular length direction. A type of vascular tomographic image is not particularly limited. For example, an ultrasound tomographic image, an optical tomographic image, or the like can be used. In addition, according to the present embodiment, a fluoroscopic image collected by the image collection unit 110 is captured while a vascular tomographic image is captured by inserting a probe into the blood vessel.

According to the present embodiment, the image collection unit 110 is connected to the tomography apparatus 170 and the fluoroscopic apparatus 180, and collects the transverse cross-sectional image and the fluoroscopic image from these apparatuses. However, the image collection unit 110 may collect the transverse cross-sectional image or the fluoroscopic image from a storage device (not illustrated) which stores the transverse cross-sectional image or the fluoroscopic image captured by the tomography apparatus 170 or the fluoroscopic apparatus 180.

For example, the ultrasound tomographic image can be acquired from an intravascular ultrasound (IVUS) apparatus or the like. In addition, for example, the optical tomographic image can be acquired from an optical coherence tomography (OCT) apparatus, an optical frequency domain imaging (OFDI) apparatus which uses wavelength sweeping, or the like. In the following description, the image collection unit 110 acquires the optical tomographic image obtained by using the OFDI apparatus.

The tomographic image acquired by the image collection unit 110 according to the present embodiment is configured to include multiple frames. For example, the transverse cross-sectional image configured to include the multiple frames can be obtained by inserting an optical probe of the OFDI apparatus into the blood vessel such as a coronary artery via a catheter and successively capturing the tomographic image while pulling back, that is, drawing back the optical probe.

In addition, a type of the vascular fluoroscopic image is also not particularly limited. For example, an X-ray image captured by applying a contrast agent using an angiography method may be used. That is, it is possible to obtain the fluoroscopic image configured to include the multiple frames by successively capturing the X-ray image while drawing back the optical probe of the OFDI apparatus. The image collection unit 110 in this embodiment therefore corresponds to an example of image collection means for collecting a vascular image extending along a vascular length direction, which is obtained from multiple transverse cross-sectional images of a blood vessel imaged by inserting a probe into the blood vessel, and multiple fluoroscopic images of the blood vessel imaged while the probe is inserted into the blood vessel.

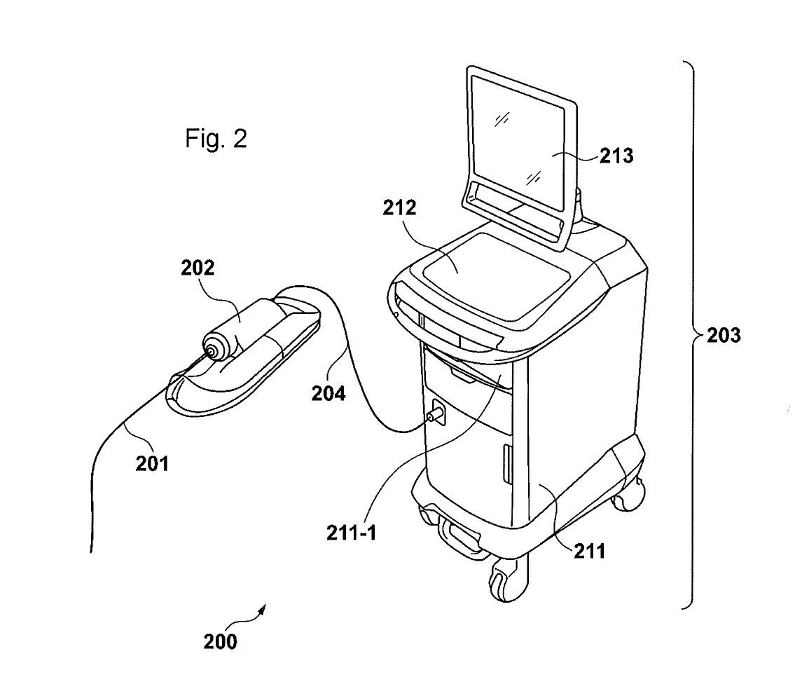

Hereinafter, the OFDI apparatus will be briefly described with reference to FIG. 2. An OFDI apparatus 200 includes a probe unit 201, a scanner and pullback unit 202, and an operation control device 203. The scanner and pullback unit 202 and the operation control device 203 are connected to each other so that various signals can be transmitted therebetween by a signal line 204.

The probe unit 201 is directly inserted into the blood vessel. An imaging core including an optical transceiver for successively transmitting transmitted light (measurement light) into the blood vessel and successively receiving reflected light from the inside of the blood vessel is internally inserted into the probe unit 201. The OFDI apparatus 200 measures an internal state of the blood vessel by applying the imaging core.

The probe unit 201 is detachably attached to the scanner and pullback unit 202. A motor incorporated therein is driven. In this manner, an axial motion and a rotational motion inside the blood vessel are regulated in the imaging core internally inserted into the probe unit 201. In addition, the scanner and pullback unit 202 acquires the reflected light received by the optical transceiver, and transmits the reflected light to the operation control device 203.

When carrying out measurement work, the operation control device 203 includes a function for inputting various set values, and a function for displaying various vascular cross-sectional images (horizontal cross-sectional image and vertical cross-sectional image) after processing data acquired by the measurement work.

The operation control device 203 includes a main body control unit 211, embodied as a processor which generates an optical cross-sectional image by processing line data generated based on the reflected light obtained by the measurement work.

A printing and storage unit 211-1, which can be a printer and DVD recorder in the embodiment, prints a processing result in the main body control unit 211, or stores the processing result as data. A user inputs various set values and instructions via an operation panel 212. An LCD monitor 211 serves as a display device which displays various cross-sectional images generated in the main body control unit 211.

The image processing apparatus 100 according to the present embodiment acquires the transverse cross-sectional image and the fluoroscopic image from an imaging device. However, the image processing apparatus 100 according to the present embodiment may be assembled to the imaging device of the transverse cross-sectional image or the fluoroscopic image. For example, the main body control unit 211 illustrated in FIG. 2 may include various configuration elements of the image processing apparatus 100 illustrated in FIG. 1. In this case, the display control unit 130 controls the display of the LCD monitor 213, and the designation acquisition unit 120 can acquire a user's instruction from the operation panel 212. In addition, an imaging system including the imaging device for capturing the transverse cross-sectional image and the imaging device for capturing the fluoroscopic image may further include the image processing apparatus 100 according to the present embodiment.

The designation acquisition unit 120 acquires the user's instruction to designate a vascular position. Specifically, the designation acquisition unit 120 acquires the user's designation to designate a first position and a second position along the vascular length direction. The designation acquisition unit 120 in this embodiment therefore corresponds to an example of designation acquisition means for acquiring a first position and a second position along the vascular length direction, which are designated by a user on the vascular image or the fluoroscopic image displayed on display means.

The display control unit 130 displays an image extending over a range in the vascular length direction on the display device 190, and a user designates the first position and the second position on the image. However, the designation acquisition unit 120 may acquire the user's designation to designate another position. For example, the user can designate a vascular position by applying an input device (not illustrated) such as a mouse. In a case where the display device 190 includes a touch screen, the user may input position designation via the touch screen. Here, the image extending over the range in the vascular length direction includes a vascular axial cross-sectional image, a vascular fluoroscopic image, and a vascular 3D image.

As described above, the display control unit 130 displays a vascular image extending along the vascular length direction, on the display device 190. Furthermore, the display control unit 130 causes the display device 190 to display the fluoroscopic image. According to the present embodiment, the display control unit 130 causes the display device 190 to display the fluoroscopic image which indicates a vascular position designated by the user's instruction. Specifically, the display control unit 130 causes the display device 190 to display the fluoroscopic image indicating the first position and the second position which are designated by the user. This process enables the user to easily recognize that the vascular position designated by the user is present at any place in the entire blood vessel. The displayed fluoroscopic image may be multiple fluoroscopic images corresponding to two points designated in the vascular length direction (axial direction of the blood vessel) and a position between the two points.

The display control unit 130 may cause the display device 190 to further display a vascular transverse cross-sectional image corresponding to the vascular position designated by the user's instruction. Specifically, the display control unit 130 can cause the display device 190 to display the transverse cross-sectional image corresponding to the first position and the transverse cross-sectional image corresponding to the second position. In this embodiment, the display control unit 130 therefore corresponds to an example of display control means for causing the display means to display an image that is designated by the user and an image that is not designated by the user from the vascular image and the fluoroscopic image which show the first position and the second position, and the display device 190 to an example of display means for displaying an image in accordance with control of the display control means.

The axial cross-sectional image and the 3D image are obtained from multiple vascular transverse cross-sectional images of the images extending over the range in the vascular length direction. Therefore, the display control unit 130 can identify the transverse cross-sectional image corresponding to the vascular position designated on the vascular image. The transverse cross-sectional image to be displayed may be multiple transverse cross-sectional images corresponding to two points designated in the vascular length direction (axial direction of the blood vessel) and a position between the two points.

The display control unit 130 can identify the fluoroscopic image captured when a probe is present near the vascular position designated by the user's instruction, with reference to a corresponding relationship acquired by the correspondence acquisition unit 140. The fluoroscopic image captured when the probe is present near the vascular position designated by the user's instruction may be a fluoroscopic image on which the image captured position of the probe is closest to the vascular position designated by the user's instruction among the multiple fluoroscopic images. Specifically, the display control unit 130 can identify the fluoroscopic image which is captured substantially concurrently with the transverse cross-sectional image corresponding to the vascular position designated by the user's instruction. The fluoroscopic image identified in this way may be a fluoroscopic image captured when the probe is present near the vascular position designated by the user's instruction. However, it is not essential to apply the correspondence acquisition unit 140. According to another embodiment, the display control unit 130 can identify a fluoroscopic image captured when the probe is present near the vascular position designated by the user's instruction, with reference to information which indicates a probe position provided on the fluoroscopic image when the fluoroscopic image is captured. The display control unit 130 can cause the display device 190 to display the fluoroscopic image indicating the first position and the second position, by applying the fluoroscopic image identified in this way.

The correspondence acquisition unit 140 acquires a corresponding relationship between each frame configuring the fluoroscopic image and each frame configuring the transverse cross-sectional image. Specifically, the correspondence acquisition unit 140 determines a frame of the fluoroscopic image captured substantially concurrently with each frame configuring the transverse cross-sectional image. A method of acquiring the corresponding relationship is not particularly limited. The frame of the fluoroscopic image captured substantially concurrently with the frame of the transverse cross-sectional image may be a frame of the fluoroscopic image captured within a predetermined period of time from the imaging time of the frame of the transverse cross-sectional image. In a case where the frame of the fluoroscopic image captured substantially concurrently is absent in the frame configuring the transverse cross-sectional image, the correspondence acquisition unit 140 can record that the frame of the corresponding fluoroscopic image is absent. According to another embodiment, the frame of the fluoroscopic image captured substantially concurrently with the frame of the transverse cross-sectional image may be a frame of the fluoroscopic image captured at the time closest to the imaged time of the frame of the transverse cross-sectional image.

As another method, the correspondence acquisition unit 140 can acquire the corresponding relationship by performing image processing on the transverse cross-sectional image and the fluoroscopic image. As an example, the correspondence acquisition unit 140 can detect a probe position from the fluoroscopic image and can calculate the inserted length of the probe so as to determine the transverse cross-sectional image corresponding to the calculated probe length. In addition, the correspondence acquisition unit 140 can acquire the corresponding relationship in accordance with a vascular bifurcated position detected from the fluoroscopic image and a vascular bifurcated position detected from the transverse cross-sectional image. Furthermore, the correspondence acquisition unit 140 can acquire the corresponding relationship with reference to a time stamp provided in each frame configuring the transverse cross-sectional image and having imaged time information and a time stamp provided in each frame configuring the fluoroscopic image. As another method, the correspondence acquisition unit 140 can acquire the corresponding relationship with reference to a frame rate of the transverse cross-sectional image and a frame rate of the fluoroscopic image.

The display control unit 130 can further cause the display device 190 to enlarge and display an image designated by the user from the images displayed on the display device 190. The image which can be enlarged is not particularly limited. The fluoroscopic image, the axial cross-sectional image, the transverse cross-sectional image, or the 3D image can be enlarged and displayed. The enlarged image may be displayed in a region where the image designated by the user is displayed, or may be displayed in another region.

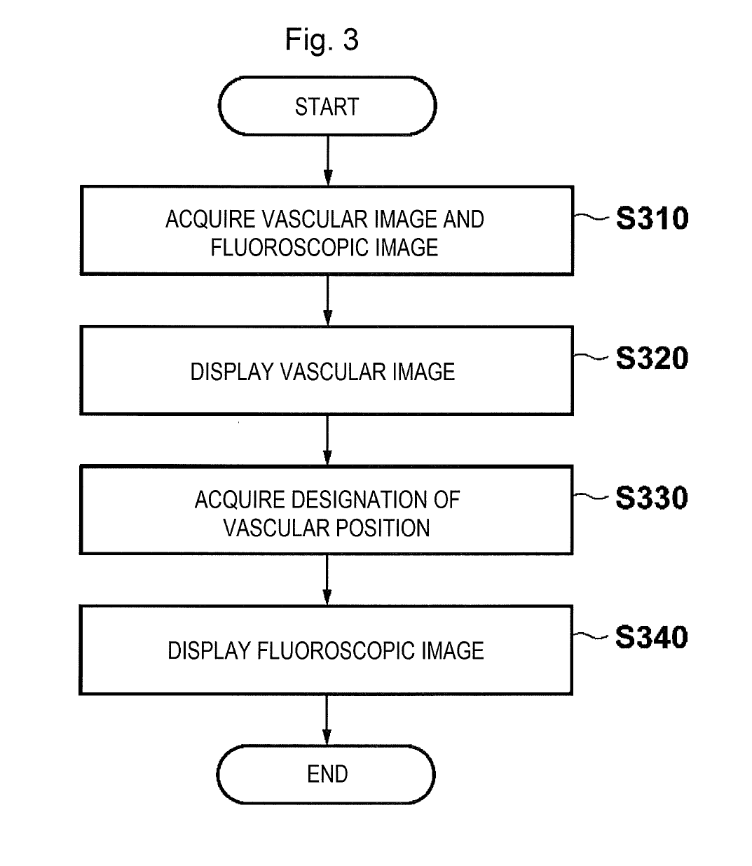

Next, a process example in an image processing method performed by the image processing apparatus 100 according to the present embodiment will be described with reference to a flowchart in FIG. 3.

In Step S310, as described above, the image collection unit 110 collects the vascular image extending along the vascular length direction and the multiple fluoroscopic images. Here, the image collection unit 110 may collect the vascular transverse cross-sectional image. In Step S320, the display control unit 130 causes the display device 190 to display the vascular image extending along the vascular length direction. In Step S330, as described above, the designation acquisition unit 120 acquires the user's instruction to designate the vascular position. In Step S340, the display control unit 130 causes the display device 190 to display the fluoroscopic image as described above in accordance with the user's instruction acquired in Step S330. Here, the display control unit 130 may cause the display device 190 to display the vascular transverse cross-sectional image.

Next, a method of acquiring the user's instruction to designate the vascular position and a method of displaying the vascular image, the fluoroscopic image, and the transverse cross-sectional image along the vascular length direction will be described in more detail with reference to the drawings. The following display examples can be appropriately switched from one to another in accordance with the user's instruction.

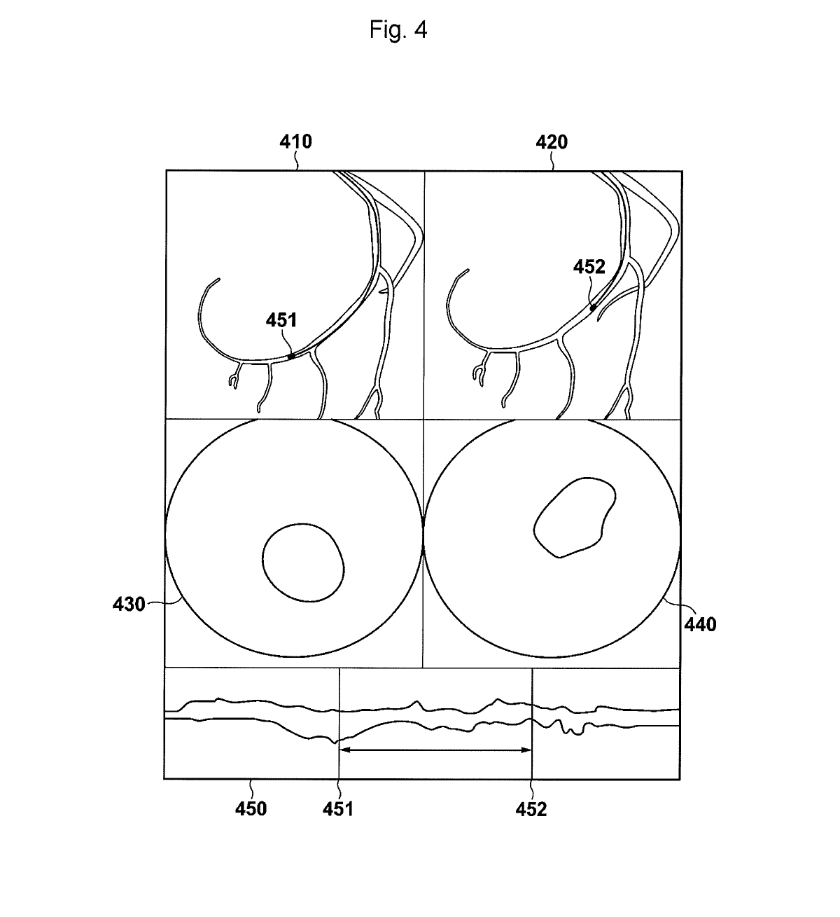

FIG. 4 illustrates a display example 1 according to the present embodiment. In the display example 1, a vascular axial cross-sectional image 450 extending along the vascular length direction is displayed on a screen of the display device 190. In the display example 1, a user designates a vascular position on the axial cross-sectional image 450. For example, the user designates a first position 451 and a second position 452 on the axial cross-sectional image 450. The designation can be performed by a click operation using a mouse, for example.

In addition, a fluoroscopic image 410 indicating the first position 451 and a fluoroscopic image 420 indicating the second position 452 are displayed on the screen of the display device 190. The fluoroscopic image 410 is captured when a probe is present near the first position 451, and the fluoroscopic image 420 is captured when the probe is present near the second position 452. A member having excellent X-ray absorbing capability is attached to a distal end of the probe. Portions absorbing much X-ray on the fluoroscopic images 410 and 420 respectively indicate the first position and the second position.

According to another embodiment, the display control unit 130 may detect the probe portion, that is, the portion absorbing much X-ray, from the fluoroscopic image 410 or 420, and may cause the display device 190 to display a marker indicating the detected portion. A type of the marker is not particularly limited. As a specific example, the display control unit 130 can superimpose a marker of a predetermined color on a probe position. In addition, the display control unit 130 can also display a superimposed triangular marker, for example, near the probe position, or can also display a superimposed circular marker surrounding the probe position, for example.

In the display example 1, a transverse cross-sectional image 430 corresponding to the first position 451 and a transverse cross-sectional image 440 corresponding to the second position 452 are further displayed on the screen of the display device 190.

The display control unit 130 may calculate the vascular length between the first position 451 and the second position 452. The display control unit 130 can cause the display device 190 to display the calculated vascular length. For example, with regard to the transverse cross-sectional image 430 corresponding to the first position 451 and the transverse cross-sectional image 440 corresponding to the second position 452, the display control unit 130 can calculate the vascular length with reference to information indicating the length for pressing the probe to a reference position. The image collection unit 110 can acquire this information together with the transverse cross-sectional image from the tomography apparatus 170. More specifically, as the vascular length, the display control unit 130 can calculate a difference between the length for pressing which corresponds to the transverse cross-sectional image 430 and the length for pressing which corresponds to the transverse cross-sectional image 440. The display control unit 130 in this embodiment therefore corresponds to an example of display control means configured to calculate a vascular length between the first position and the second position and cause the display means to display the calculated vascular length.

However, a method of calculating the vascular length is not limited to this method. For example, in a case where the transverse cross-sectional image is captured at a constant interval, it is possible to calculate the vascular length, based on a difference between the frame number of the transverse cross-sectional image 430 and the frame number of the transverse cross-sectional image 440. In this case, the image collection unit 110 can acquire information indicating the vascular length between imaging positions of the successive frames together with a tomographic image from the tomography apparatus 170.

A method of designating the first position 451 and the second position 452 is not limited to the method of designating the first position 451 and the second position 452 by performing the click operation. For example, the first position and the second position can also be moved by performing a drag operation. In addition, a user can also pre-designate the vascular length between the first position 451 and the second position 452. In this case, the display control unit 130 moves both the first position 451 and the second position 452 according to the user's movement instruction so that the vascular length between the first position 451 and the second position 452 becomes the length designated by the user. For example, through the drag operation, the user can concurrently move both the first position 451 and the second position 452 so that the vascular length between the first position 451 and the second position 452 is not changed. This configuration is advantageously adopted in order to search for a position suitable for stent indwelling in which a stent length is determined in advance. The display control unit in this embodiment therefore corresponds to an example of display control means for moving both the first position and the second position in accordance with a user's movement instruction so that the vascular length between the first position and the second position becomes a length designated by the user's instruction.

In the display example 1, hereinafter, a process performed by the display control unit 130 will be described in detail. If the user designates the vascular position, the fluoroscopic images 410 and 420 and the transverse cross-sectional images 430 and 440 which correspond to the vascular position are displayed on the display device 190. Specifically, the display control unit 130 acquires the transverse cross-sectional image 430 corresponding to the first position 451 and the transverse cross-sectional image 440 corresponding to the second position 452, from the image collection unit 110. Furthermore, the display control unit 130 acquires the fluoroscopic image 410 captured when the probe is present near the first position and the fluoroscopic image 420 captured when the probe is present near the second position, from the image collection unit 110. A method of identifying the acquired transverse cross-sectional images 430 and 440 and the acquired fluoroscopic images 410 and 420 is employed as described above.

According to the display example 1, it is possible to easily recognize two positions on the fluoroscopic image which are designated on the vascular axial cross-sectional image. For example, this display is advantageously used in order to determine an indwelling position of the stent. It is preferable to cause the stent to indwell so that an end portion thereof is not located in a vascular bifurcated portion. In addition, it is preferable to cause the stent to indwell a vascular hardened portion. As described above, the display according to the present embodiment is advantageously used particularly in order to determine the vascular position where the end portion of the stent is arranged. In addition, since it is possible to easily recognize the position on the fluoroscopic image of the vascular position designated by the user, the user can more easily cause the stent to indwell at a desired position while checking the fluoroscopic image.

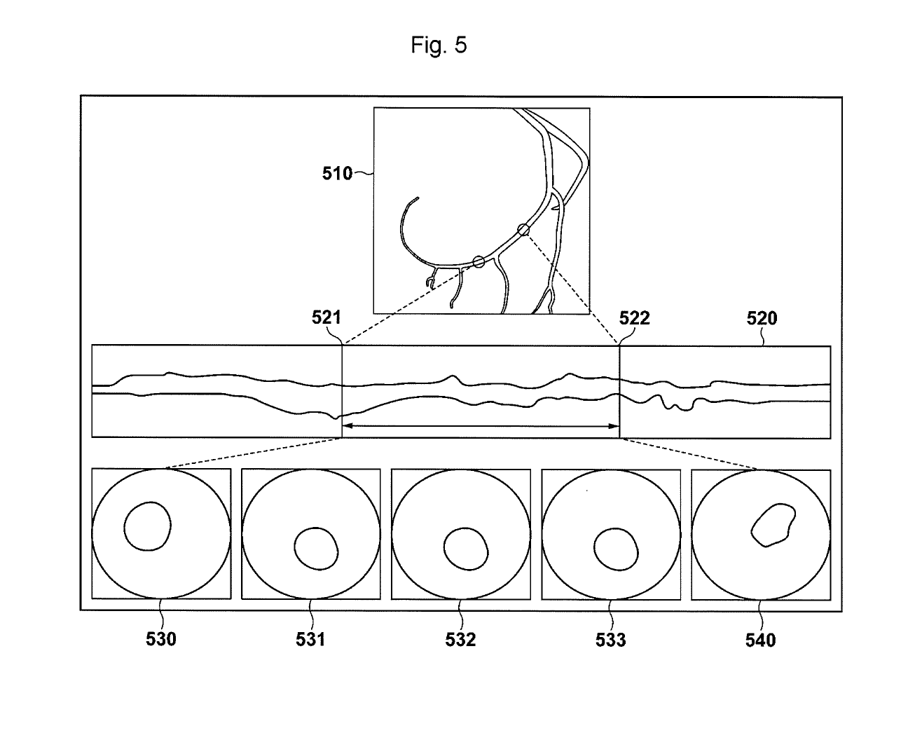

FIG. 5 illustrates a display example 2 according to the present embodiment. In the display example 2, similarly to the display example 1, a vascular axial cross-sectional image 520 is displayed on the screen of the display device 190. A user can designate a first position 521 and a second position 522 on the axial cross-sectional image 520. In addition, a fluoroscopic image 510 for indicating the first position 521 and the second position 522 is displayed on the screen of the display device 190. The fluoroscopic image 510 is captured when the probe is present near the second position 522. The first position 521 and the second position 522 are displayed on the fluoroscopic image 510 by using a marker. A type of the marker is not particularly limited. For example, the first position 521 and the second position 522 can be displayed by using the marker described in the display example 1.

In the display example 2, the first position 521 and the second position 522 are displayed on one fluoroscopic image 510. However, similarly to the display example 1, the fluoroscopic image indicating the first position 521 and the fluoroscopic image indicating the second position 522 may be concurrently displayed on the display device 190. In addition, the fluoroscopic image 510 may be captured when the probe is present near the first position 521. Furthermore, as long as the fluoroscopic image 510 indicates the first position 521 and the second position 522, any desired fluoroscopic image may be employed. For example, the fluoroscopic image may be captured when the probe is present in the middle between the first position 521 and the second position 522.

In the display example 2, a transverse cross-sectional image 530 corresponding to the first position 521 and a transverse cross-sectional image 540 corresponding to the second position 522 are displayed. In addition, in the display example 2, vascular transverse cross-sectional images 531 to 533 at the vascular position between the first position and the second position are concurrently displayed on the display device 190. A method of determining the transverse cross-sectional images 531 to 533 to be displayed is not particularly limited. For example, the transverse cross-sectional images at respective positions determined so as to have an equal interval between the first position and the second position may be displayed on the display device 190.

In addition, the display control unit 130 may cause the display device 190 to display markers indicating the vascular positions corresponding to the transverse cross-sectional images 531 to 533, on the axial cross-sectional image 520. Furthermore, the display control unit 130 may cause the display device 190 to display the fluoroscopic image indicating the vascular position corresponding to the transverse cross-sectional images 531 to 533. Specifically, the display control unit 130 can cause the display device 190 to display each fluoroscopic image captured when the probe is present at the vascular position corresponding to the transverse cross-sectional images 531 to 533. In addition, the display control unit 130 can also cause the display device 190 to display markers indicating the vascular positions corresponding to the transverse cross-sectional images 531 to 533, on any desired fluoroscopic image, for example, on the fluoroscopic image 510.

According to another embodiment, at least one of one or more transverse cross-sectional images displayed in addition to the transverse cross-sectional images at the first position 521 and the second position 522 is a transverse cross-sectional image at a position where it is determined, by the display control unit 130, that a diameter or a cross-sectional area of a vascular lumen is smallest. For example, the vascular diameter and the vascular cross-sectional area can be calculated by extracting an intravascular wall portion from each transverse cross-sectional image. A type of the vascular diameter is not particularly limited. For example, the type may be the smallest lumen diameter or the largest lumen diameter. At this time, the vascular diameter can be calculated with reference to resolution information provided on a tomographic image acquired by the image collection unit 110. The display control unit 130 in this embodiment therefore corresponds to an example of display control means which detects a third position where a diameter or a cross-sectional area of a lumen of the blood vessel is smallest between the first position and the second position, and causes the display means to further display the transverse cross-sectional image at the third position.

In the display example 2, hereinafter, a process performed by the display control unit 130 will be described in detail. If a user designates a vascular position, the fluoroscopic image 510 and the transverse cross-sectional images 530 and 540 are displayed on the display device 190. Specifically, similarly to the display example 1, the display control unit 130 acquires the transverse cross-sectional image 530 corresponding to the first position 521 and the transverse cross-sectional image 540 corresponding to the second position 522, from the image collection unit 110. Furthermore, the display control unit 130 acquires the fluoroscopic image 510 which is optionally selected as described above, from the image collection unit 110.

Furthermore, the display control unit 130 displays markers indicating the first position 521 and the second position 522, on the fluoroscopic image 510. According to an embodiment, coordinates of the probe portion on the fluoroscopic image, which is detected from the fluoroscopic image captured when the probe is present near the first position 521 or the second position 522, are used as coordinates of the first position 521 or the second position 522 on the fluoroscopic image 510. According to another embodiment, a configuration may be adopted so as to compensate for misalignment in the vascular positions between the fluoroscopic image 510 and a fluoroscopic image A captured when the probe is present near the first position 521 or the second position 522. Specifically, it is possible to detect the position on the fluoroscopic image 510, which corresponds to the probe position on the fluoroscopic image A, by superimposing the fluoroscopic image 510 and the fluoroscopic image A on each other so that the vascular positions are superimposed on each other.

In the display example 2, the transverse cross-sectional image at the first position and the second position and one or more transverse cross-sectional images present between the first position and the second position are displayed. Referring to the transverse cross-sectional images, a user more easily recognizes a vascular state between the first position and the second position.

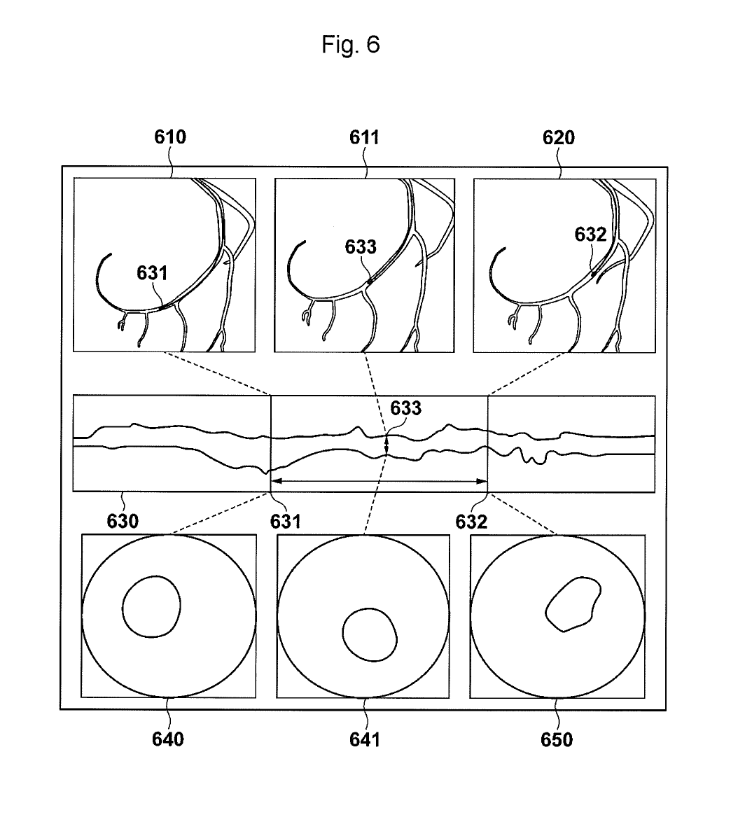

FIG. 6 illustrates a display example 3 according to the present embodiment. In the display example 3, an axial cross-sectional image 630 is displayed on the screen of the display device 190. A user can designate a first position 631 and a second position 632 on the axial cross-sectional image 630. In addition, a fluoroscopic image 610 indicating the first position 631, a fluoroscopic image 620 indicating the second position 632, a transverse cross-sectional image 640 corresponding to the first position 631, and a transverse cross-sectional image 650 corresponding to the second position 632 are displayed on the screen of the display device 190. In addition, the display is the same as that in the display example 1.

A transverse cross-sectional image 641 at a position 633 where the diameter or the cross-sectional area of the vascular lumen is smallest between the first position 631 and the second position 632 is further displayed on the screen of the display device 190. The position 633 can be determined by using a method described in the display example 2. A marker indicating the determined position 633 is displayed on the axial cross-sectional image 630. In addition, a fluoroscopic image 611 indicating the determined position 633 is displayed on the screen of the display device 190. The fluoroscopic image 611 is captured when the probe is present near the position 633, and can be identified by using the above-described method.

A vascular diameter or a vascular cross-sectional area at the position 633 is further displayed on the screen of the display device 190. The vascular diameter or the vascular cross-sectional area can be measured by a method described in the display example 2. A method of displaying the vascular diameter or the vascular cross-sectional area is not particularly limited. Any one of these may be superimposed and displayed near the position 633 on the axial cross-sectional image 630, or may be displayed in a region separate from the axial cross-sectional image 630.

The display control unit 130 can calculate not only the vascular diameter or the vascular cross-sectional area at the position 633 but also any desired information relating to the blood vessel, and can cause the display device 190 to display the information. For example, the display control unit 130 can calculate a statistic value of the cross-sectional area or the diameter of the vascular lumen between the first position 631 and the second position 632. For example, the statistic value includes the maximum value, the minimum value, and an average value. The display control unit 130 in this embodiment therefore corresponds to an example of display control means configured to calculate a statistic value of the diameter or the cross-sectional area of a lumen of the blood vessel between the first position and the second position and cause the display means to display the calculated vascular length.

The marker indicating the position 633, the transverse cross-sectional image 641 at the position 633, and the fluoroscopic image 611 indicating the position 633 may be updated as the first position 631 or the second position 632 is moved in accordance with the user's designation.

In the display example 3, a stenosis area between the first position and the second position is automatically displayed. Accordingly, the user more easily recognizes a vascular state between the first position and the second position. For example, this display is advantageously used in order to select a stent which indwells between the first position and the second position.

According to another embodiment, instead of displaying the transverse cross-sectional image 641, multiple vascular transverse cross-sectional images at a vascular position between the first position 631 and the second position 632 are displayed while being sequentially switched to each other. In other words, the transverse cross-sectional images sequentially captured at the vascular position between the first position and the second position are displayed as a moving image. At this time, it is also possible to display a marker indicating the vascular position on the axial cross-sectional image 630, which corresponds to the displayed transverse cross-sectional image. In addition, it is also possible to sequentially display the fluoroscopic images indicating the vascular position corresponding to the displayed transverse cross-sectional image.

According to further another embodiment, with regard to multiple vascular positions between the first position 631 and the second position 632, the fluoroscopic images indicating the respective vascular positions are displayed while being sequentially switched to each other. In other words, the fluoroscopic images sequentially captured when the probe is moved (is pulled back) between the first position and the second position are displayed as the moving image. At this time, it is also possible to display a marker indicating the vascular position on the axial cross-sectional image 630, which corresponds to the displayed fluoroscopic image. In this case, it is not essential to designate the first position and the second position and to display the fluoroscopic images 610 and 620. All frames of the captured fluoroscopic image may be sequentially displayed. According to this configuration, it is possible to easily recognize that each vascular position on the vascular image extending along the vascular length direction corresponds to any position on the fluoroscopic image. In addition, it is not essential to display the transverse cross-sectional images 640 and 650 or the transverse cross-sectional image of the vascular position indicated by the displayed fluoroscopic image. However, if these are displayed, a user more easily recognizes a vascular shape.

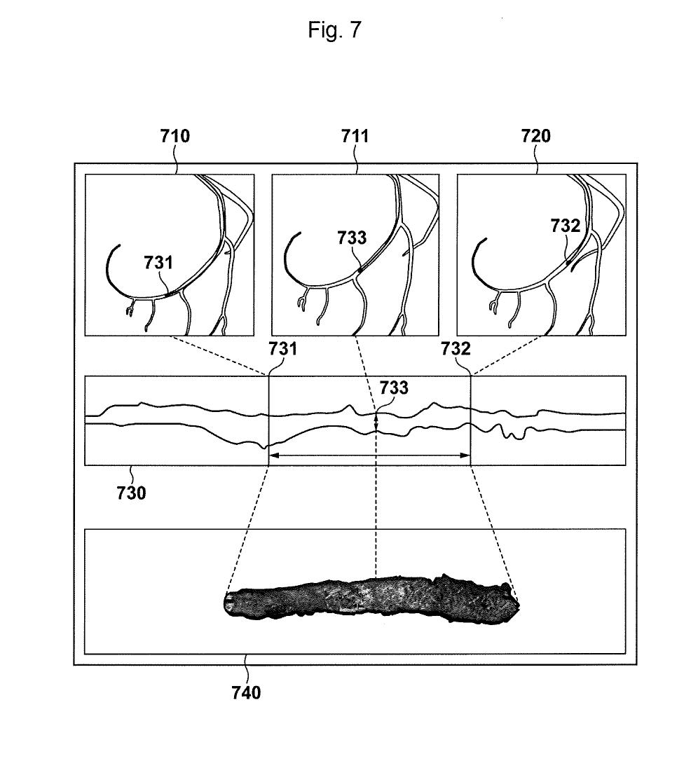

FIG. 7 illustrates a display example 4 according to the present embodiment. In the display example 4, an axial cross-sectional image 730 is displayed on the screen of the display device 190. A user can designate a first position 731 and a second position 732 on the axial cross-sectional image 730. In addition, a fluoroscopic image 710 indicating the first position 731 and a fluoroscopic image 720 indicating the second position 732 are displayed on the screen of the display device 190. Furthermore, a position 733 where the vascular diameter or the vascular cross-sectional area is smallest between the first position 731 and the second position 732, and a fluoroscopic image 711 captured when the probe is present near the position 733 are displayed on the screen of the display device 190. The display is the same as that in the display example 3.

In the display example 4, a 3D image 740 of the blood vessel between the first position 731 and the second position 732 is further displayed on the screen of the display device 190. As described above, the 3D image 740 can be reconstructed to include a tomographic image by the display control unit 130. A marker indicating the position 733 where the vascular diameter or the vascular cross-sectional area is smallest on the 3D image 740 may be further displayed on the screen of the display device 190.

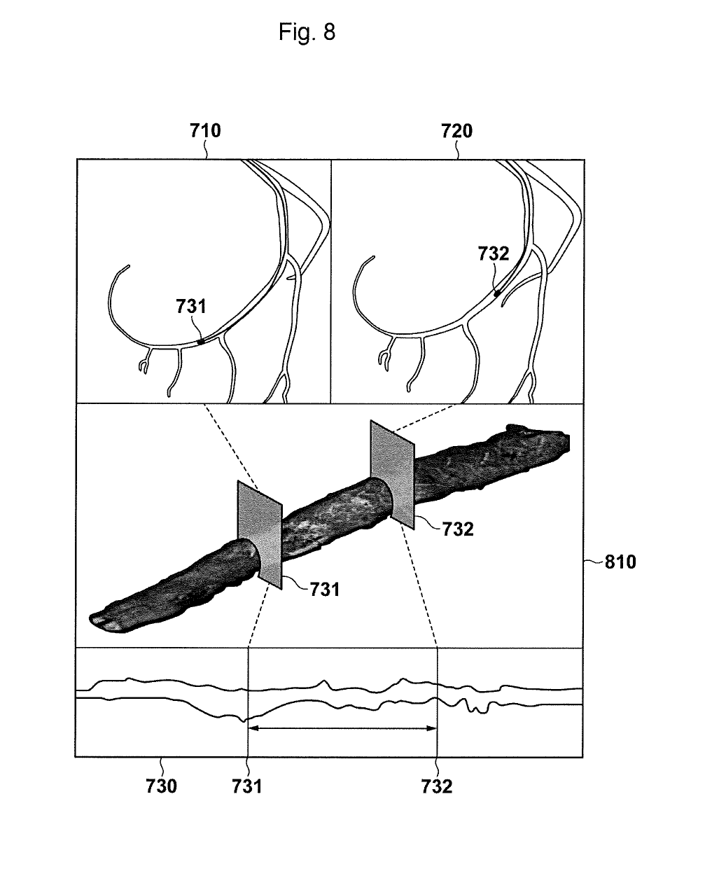

In addition, as illustrated in FIG. 8, instead of displaying the 3D image 740 of the blood vessel between the first position 731 and the second position 732, a 3D image 810 of the blood vessel including the first position 731 and the second position 732 may be displayed. In this case, a marker indicating the first position 731 and a marker indicating the second position 732 can be displayed on the 3D image 810. The 3D image 810 displayed in this way also shows the vascular image extending along the vascular length direction. That is, according to further another embodiment, a user can designate the first position 731 and the second position 732 on the 3D image 810.

In the display example 4, the 3D image between the first position and the second position is automatically displayed. Accordingly, the user more easily recognizes a vascular state between the first position and the second position.

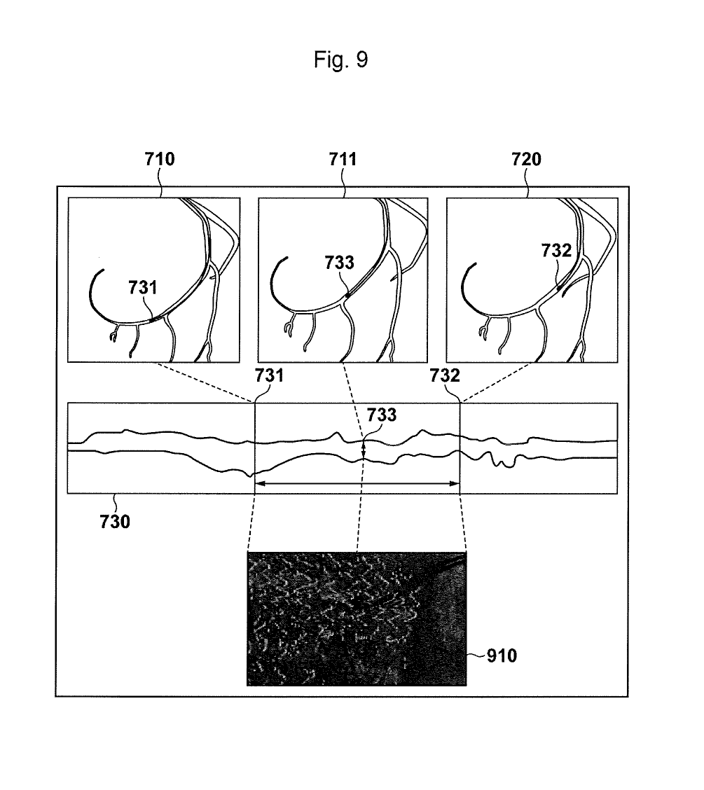

FIG. 9 illustrates a display example 5 according to the present embodiment. The display example 5 is similar to the display example 4. However, instead of the 3D image 740 of the blood vessel, a 3D image 910 which is a deployment view of the blood vessel between the first position 731 and the second position 732 is displayed. Similarly to the 3D image 740, the 3D image 910 can be reconstructed to include the vascular transverse cross-sectional image. In this display example, a user also more easily recognizes a vascular state between the first position and the second position.

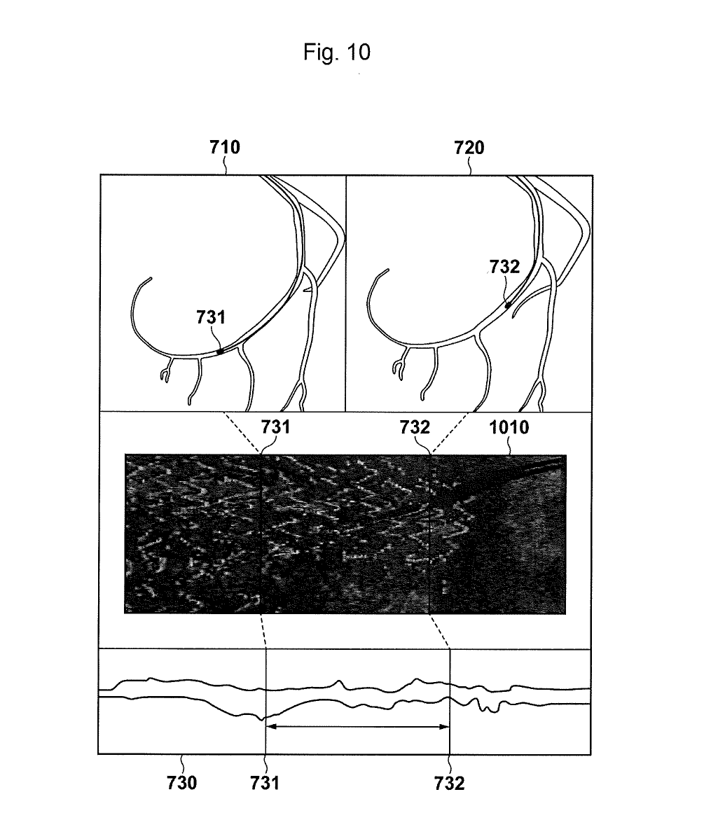

In addition, as illustrated in FIG. 10, instead of displaying the 3D image 910 which is a deployment view of the blood vessel between the first position 731 and the second position 732, a 3D image 1010 which is a deployment view of the blood vessel including the first position 731 and the second position 732 may be displayed. In this case, a marker indicating the first position 731 and a marker indicating the second position 732 can be displayed on the 3D image 1010. The 3D image 1010 displayed in this way also shows the vascular image extending along the vascular length direction. That is, according to further another embodiment, the user can designate the first position 731 and the second position 732 on the 3D image 1010.

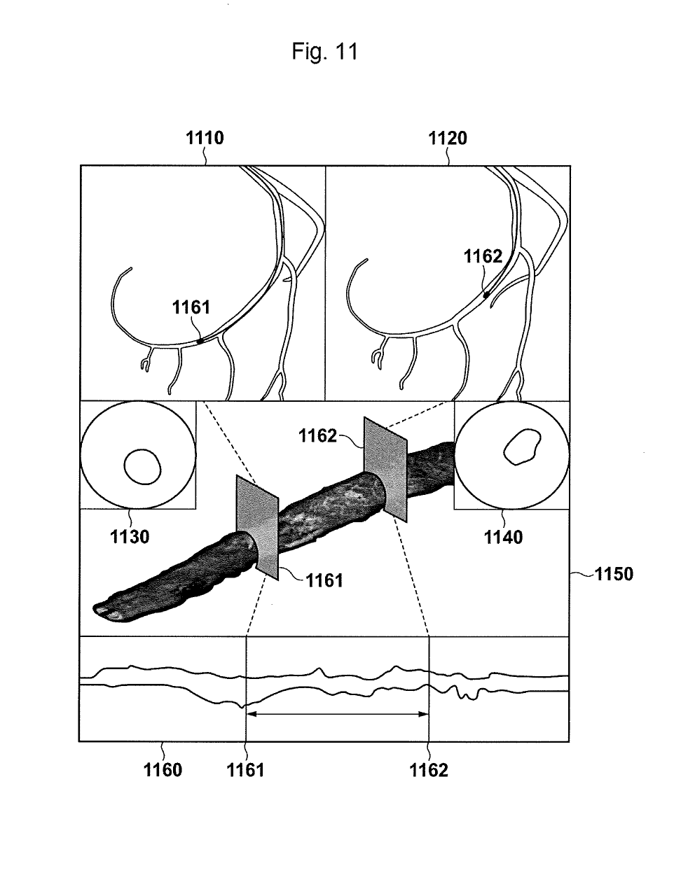

FIG. 11 illustrates a display example 6 according to the present embodiment. In the display example 6, an axial cross-sectional image 1160 is displayed on the screen of the display device 190. A user can designate a first position 1161 and a second position 1162 on the axial cross-sectional image 1160. In addition, a fluoroscopic image 1110 indicating the first position 1161 and a fluoroscopic image 1120 indicating the second position 1162 are displayed on the screen of the display device 190. Furthermore, a transverse cross-sectional image 1130 corresponding to the first position 1161 and a transverse cross-sectional image 1140 corresponding to the second position 1162 are displayed on the screen of the display device 190. A 3D image 1150 of the blood vessel is displayed on the screen of the display device 190, and a marker indicating the first position 1161 and a marker indicating the second position 1162 are displayed on the 3D image 1150. In this display example, the user also more easily recognizes a vascular state between the first position and the second position.

The above-described respective embodiments can also be realized by a computer-readable program that causes a computer to execute processes. That is, the computer-readable program for realizing a function of each unit according to the above-described respective embodiments is supplied to a system or an apparatus which includes the computer via a network or a storage medium. Then, the computer including a processor and a memory causes the memory to read the computer-readable program, and the processor is operated in accordance with the computer-readable program read on the memory, thereby enabling the computer to realize the above-described respective embodiments. The program can be stored on a tangible, non-transitory computer readable storage medium, such as a memory, a hard disk, a CD-ROM, and the like.

A user can also designate a fluoroscopic image. For example, the display control unit 130 can display the fluoroscopic image on the display device 190. The designation acquisition unit 120 can acquire the first position and the second position along the vascular length direction, which are designated by the user on the fluoroscopic image displayed on the display device 190. In this case, the display control unit 130 can cause the display device 190 to display a vascular image, which is not used in designating positions by the user between the vascular image and the fluoroscopic image, and which indicates the first position and the second position.

The detailed description above describes an image processing apparatus, image display system, imaging system, image processing method, and program. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing apparatus comprising:
image collection means for collecting a vascular image extending along a vascular length direction, which is obtained from multiple transverse cross-sectional images of a blood vessel imaged by inserting a probe into the blood vessel, and multiple fluoroscopic images of the blood vessel imaged while the probe is inserted into the blood vessel;
designation acquisition means for acquiring a first position and a second position along the vascular length direction, which are designated by a user on the vascular image or the fluoroscopic image displayed on display means; and
display control means for causing the display means to display an image that is designated by the user and an image that is not designated by the user from the vascular image and the fluoroscopic image which show the first position and the second position.

2. The image processing apparatus according to claim 1,
wherein the vascular image is an axial cross-sectional image of the blood vessel.

3. The image processing apparatus according to claim 1,
wherein the display control means causes the display means to further display a transverse cross-sectional image of the blood vessel at the first position and a transverse cross-sectional image of the blood vessel at the second position.

4. The image processing apparatus according to claim 1,
wherein the display control means causes the display means to display the fluoroscopic image captured when the probe is present near the first position and the fluoroscopic image captured when the probe is present near the second position, as the fluoroscopic image which shows the first position and the second position.

5. The image processing apparatus according to claim 1,
wherein the display control means displays markers indicating the first position and the second position on the fluoroscopic image.

6. The image processing apparatus according to claim 1,
wherein the display control means causes the display means to further display a 3D image of the blood vessel, and
wherein the 3D image of the blood vessel is either a 3D image of the blood vessel from the first position to the second position or a 3D image of the blood vessel which is provided with markers indicating the first position and the second position.

7. The image processing apparatus according to claim 1, wherein the display control means detects a third position where a diameter or a cross-sectional area of a lumen of the blood vessel is smallest between the first position and the second position, and causes the display means to further display the transverse cross-sectional image at the third position.

8. The image processing apparatus according to claim 1,
wherein the display control means causes the display means to further display the multiple transverse cross-sectional images at multiple third positions between the first position and the second position while the multiple transverse cross-sectional images are switched from one to another.

9. The image processing apparatus according to claim 1,
wherein the display control means causes the display means to further concurrently display one or more transverse cross-sectional images at one or more third positions between the first position and the second position.

10. The image processing apparatus according to claim 7,
wherein the display control means causes the display means to display a marker indicating the third position on the vascular image.

11. The image processing apparatus according to claim 7,
wherein the display control means causes the display means to display the fluoroscopic image which shows the third position.

12. The image processing apparatus according to claim 1, wherein the display control means is further configured to calculate a vascular length between the first position and the second position and cause the display means to display the calculated vascular length.

13. The image processing apparatus according to claim 1, wherein the display control means is further configured to calculate a statistic value of the diameter or the cross-sectional area of a lumen of the blood vessel between the first position and the second position and cause the display means to display the calculated statistic value of the diameter or the cross-sectional area.

14. The image processing apparatus according to claim 1, wherein the display control means is further configured to move both the first position and the second position in accordance with a user's movement instruction so that the vascular length between the first position and the second position becomes a length designated by the user's instruction.

15. The image processing apparatus according to claim 1,
wherein the display control means causes the display means to enlarge and display an image designated by the user from the images displayed on the display means.

16. An image display system comprising:
the image processing apparatus according to claim 1; and
display means for displaying an image in accordance with control of the display control means.

17. A imaging system comprising:
the image processing apparatus according to claim 1;
a tomography apparatus that acquires the vascular transverse cross-sectional images to be collected by the image processing apparatus; and
a fluoroscopic apparatus that acquires the vascular fluoroscopic images to be collected by the image processing apparatus.

18. An image processing method performed by an image processing apparatus, comprising:
an image collection step of collecting a vascular image extending along a vascular length direction, which is obtained from multiple transverse cross-sectional images of a blood vessel imaged by inserting a probe into the blood vessel, and multiple fluoroscopic images of the blood vessel imaged while the probe is inserted into the blood vessel;
a first display control step of causing display means to display the vascular image;
a designation acquisition step of acquiring a first position and a second position along the vascular length direction, which are designated by a user on the vascular image or the fluoroscopic image displayed on the display means; and
a second display control step of causing the display means to display an image that is designated by the user and an image that is not designated by the user from the vascular image and the fluoroscopic image which show the first position and the second position.

19. An imaging system, comprising:
a tomography apparatus that acquires vascular transverse cross-sectional images;
a fluoroscopic apparatus that acquires vascular fluoroscopic images;
a display for displaying an image; and
a processor configured to:
collect a vascular image extending along a vascular length direction, which is obtained from multiple transverse cross-sectional images of a blood vessel imaged by the tomography apparatus, and multiple fluoroscopic images of the blood vessel imaged by the fluoroscopic apparatus,
acquire a first position and a second position along the vascular length direction, which are designated by a user on the vascular image or the fluoroscopic image displayed on the display, and
cause the display to display an image that is designated by the user and an image that is not designated by the user from the vascular image and the fluoroscopic image which show the first position and the second position.
